(19) United States
(12) Patent Application Publication
(10) Pub. No.: US 2020/0222569 A1
(43) Pub. Date: Jul. 16, 2020

(54) METHODS AND TEST KITS FOR ASSESSING HAND HYGIENE

(71) Applicants: Standard Textile Co., Inc., Cincinnati, OH (US); Q Laboratories, Inc., Cincinnati, OH (US)

(72) Inventors: Bradley J. Bushman, Cincinnati, OH (US); Patrick Bird, West Chester, OH (US); Dane Brooks, Newport, KY (US)

(21) Appl. No.: 16/249,231

(22) Filed: Jan. 16, 2019

(51) Int. Cl. A61L 2/28; C12Q 1/24; A61L 2/00; B08B 3/08

(52) U.S. Cl. CPC A61L 2/28; C12Q 1/24; A41D 19/0055; B08B 3/08; A61L 2202/17; A61L 2/0088

(57) ABSTRACT

Methods and test kits are provided for assessing effectiveness of an individual's hand cleansing technique. One method includes donning a glove on one hand and another glove on the other, which then are engaged for a desired period of time. The gloves are removed and the hands cleansed. Thereafter, a third glove is donned on one hand and a fourth glove on the other, which again are engaged. Alternatively or additionally, at least one fingertip on one hand optionally can be swabbed with a swab device to collect a sample from the fingertip, then the hands cleansed. At least one cleansed fingertip can be swabbed with a swab device to collect a sample. The gloves and/or test swab(s) can be sent to a lab and tested for a microbe presence to assesses effectiveness of an individual's hand cleansing technique. Kits can be provided to aid in carrying out said methods.

### TECHNICAL FIELD

The present invention relates to hand hygiene and, in particular, to methods and test kits for assessing hand hygiene, including the effectiveness of an individual's hand cleansing technique.

### BACKGROUND

Proper hand hygiene and hand cleansing has been identified as a critical factor in preventing infections and the spread thereof, particularly in healthcare facilities, such as hospitals. On any given day, about 1 in 25 hospital patients has at least one healthcare-associated infection. Practicing good hand hygiene is a simple, yet effective, way to prevent such infections. That is, properly cleansing hands can help prevent the spread of germs, including those that are resistant to antibiotics and becoming difficult, if not impossible, to treat.

Textile service providers and laundries, particularly their employees, have a responsibility to maintain proper hand hygiene especially when handling and/or transporting laundered textile products including linens and gowns, such as for use by patients and healthcare workers in healthcare facilities. Proper hand hygiene and hand cleansing is even more of a concern for those employees who may travel or work between the soiled side of the laundry (contaminated textiles) and the clean side. Washing hands or use of alcohol-based hand sanitizers, for example, is required for this and generally when individuals start work, return from break, and return from lunch or if hands have been visibly soiled. Proper hand cleansing techniques are paramount to ensuring an acceptable level of hand hygiene to reduce or eliminate the potential spread of contaminates or pathogens via textile products, such as laundered linens and gowns for use in healthcare facilities.

It would be beneficial to provide a method and kit for testing an individual's hand hygiene, such as an employee of a textile service provider or laundry facility that handles and/or transports laundered textile products including linens and gowns, such as for use by patients and healthcare workers in healthcare facilities, as a way to assess the effectiveness of that individual's hand cleansing technique(s).

### SUMMARY OF THE INVENTION

The present invention provides for a method and kit for testing an individual's hand hygiene to assess the effectiveness of that individual's hand cleansing technique.

In one embodiment, a method for assessing the effectiveness of an individual's hand cleansing technique is provided and includes donning a first glove on an individual's hand and then engaging the gloved hand and a second hand of the individual for a desired period of time. The method then includes removing the first glove from the hand and, after removing the first glove, cleansing the hand. Thereafter, a second glove is donned on the cleansed hand and the gloved hand having the second glove and the second hand of the individual are engaged for a desired period of time. Next, the second glove is removed from the hand, and wherein an interior of the first glove and the second glove is ready to be tested for a microbe presence so that test data can be evaluated to assess the effectiveness of the individual's hand cleansing technique. In one example, after cleansing the hand, at least one fingertip on the individual's cleansed hand is swabbed with a first swab device to collect a first sample from the fingertip, wherein the first sample is ready to be tested for a microbe presence so that test data can be evaluated to assess the effectiveness of the individual's hand cleansing technique.

In another embodiment, a method for assessing the effectiveness of an individual's hand cleansing technique is provided and includes donning a first glove on an individual's first hand and a second glove on a second hand of the individual and then engaging the gloved first hand and the gloved second hand for a desired period of time. The method then includes removing the first and second gloves from the first and second hands and, after removing the first and second gloves, cleansing the first and second hands. Thereafter, a third glove on the first cleansed hand is donned and a fourth glove on the second cleansed hand is donned. Then, the gloved first hand having the third glove and the gloved second hand having the fourth glove are engaged for a desired period of time. Next, the third and fourth gloves are removed from the first and second hands, wherein an interior of at least one of the first or second gloves and one of the third or fourth gloves is ready to be tested for a microbe presence so that test data can be evaluated to assess the effectiveness of the individual's hand cleansing technique. In one example, after cleansing the first and second hands, at least one fingertip on the individual's cleansed first hand is swabbed with a first swab device to collect a first sample from the fingertip and at least one fingertip on the individual's cleansed second hand is swabbed with a second swab device to collect a second sample from the fingertip, wherein the first and second samples are ready to be tested for a microbe presence so that test data can be evaluated to assess the effectiveness of the individual's hand cleansing technique.

In another embodiment, a method for assessing the effectiveness of an individual's hand cleansing technique is provided and includes swabbing at least one fingertip on an individual's first hand with a first swab device to collect a first sample from the fingertip and then cleansing the first hand of the individual. Thereafter, at least the same fingertip on the individual's cleansed first hand is swabbed with a second swab device to collect a second sample from the fingertip, wherein the first and second sample of the first and second swab devices are ready to be tested for a microbe presence so that test data can be evaluated to assess the effectiveness of the individual's hand cleansing technique.

In yet another embodiment, a test kit for assessing the effectiveness of an individual's hand cleansing technique is provided and includes at least two pairs of gloves, with one glove from each pair capable of being donned on an individual's left hand and the other glove capable of being donned on the individual's right hand, and/or at least a first and second swab device, with each swab device configured to swab at least one fingertip on an individual's first hand and collect a sample from the fingertip. The test kit further includes one or more containers or packages for receiving the gloves and/or swab devices after use by the individual, wherein the gloves and/or swab devices can be sent to a testing facility for testing to assess the effectiveness of an individual's hand cleansing technique. In one example, the test kit comprises three pairs of gloves, a first, second, and third swab device, and four containers or packages.

### BRIEF DESCRIPTION OF THE DRAWINGS

The accompanying drawings, which are incorporated in and constitute a part of this specification, illustrate embodiments of the invention and, with a detailed description of the embodiments given below, serve to explain the principles of the invention. The components in the figures are not necessarily to scale. Moreover, in the figures, like-referenced numerals designate corresponding parts throughout the different views.

FIG. 1 is a flowchart of a method for testing an individual's hand cleansing effectiveness in accordance with an embodiment of the invention;

FIG. 2 is a flowchart of a method for testing an individual's hand cleansing effectiveness in accordance with another embodiment of the invention;

FIG. 3 shows a gloved first hand and a gloved second hand engaged, which may result in a deposit of microbes on an interior surface of the gloves in accordance with certain steps of the test method of FIG. 1;

FIG. 4 shows a glove filled with an extraction medium to extract a microbe from an interior surface of a glove of FIG. 3 to test for microbe presence;

FIG. 5 shows a fingertip and area under a finger nail being swabbed via a swab device to collect microbes onto the swab device in accordance with certain steps of the test method of FIG. 2; and

FIG. 6 shows a kit for testing an individual's hand cleansing effectiveness in accordance with an embodiment of the invention.

### DETAILED DESCRIPTION

Methods of testing the effectiveness of a person's hand cleansing technique(s), e.g., hand washing techniques and/or use of alcohol-based gels/liquids or other disinfectants, are provided herein and discussed in detail hereinbelow. In some examples, individual test subjects, e.g., an employee of a textile service provider or laundry facility that handles and/or transports laundered textile products including linens and gowns such as for use by patients and healthcare workers in healthcare facilities, may not be told ahead of time when or where testing will take place to ensure that the individual does not alter his/her behavior prior to said test. Such testing may reveal deficiencies in one's hand cleansing technique. Also, certain pathogens or contaminates may be revealed as present in an individual's environment. Certain pathogens or contaminates also may be revealed as especially difficult to remove from one's hands without proper technique(s).

For clarity, as used herein, the term “finger” includes a thumb. In addition, the term “first hand” refers to either the right or left hand of a person, while the term “second hand” refers to the other of the right or left hand of a person that is not the first hand. Though the figures show the first hand as being a left hand, this is not meant to be limiting as to which hand, right or left, is considered the first hand when practicing the methods and utilizing the kit described herein.

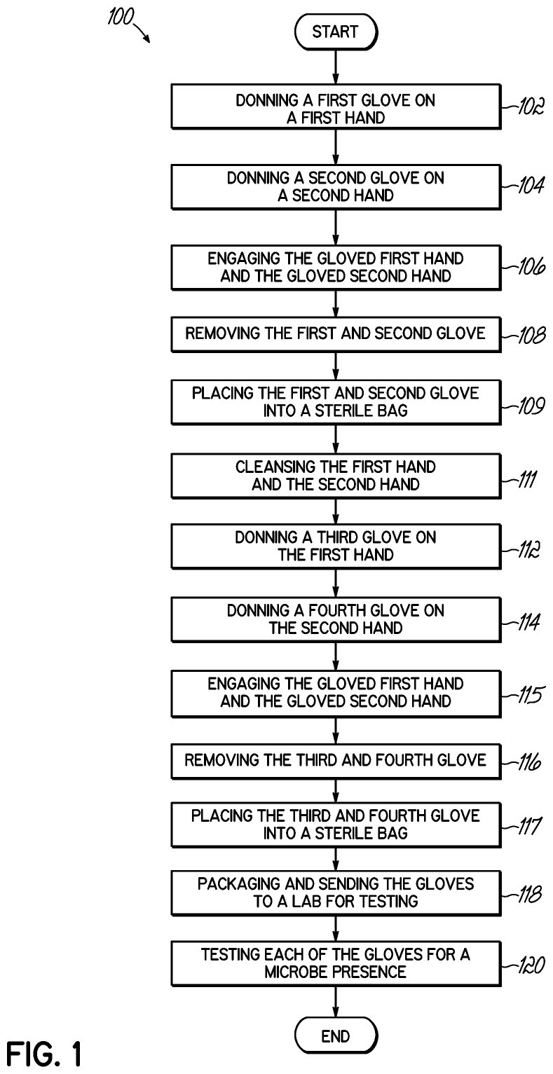

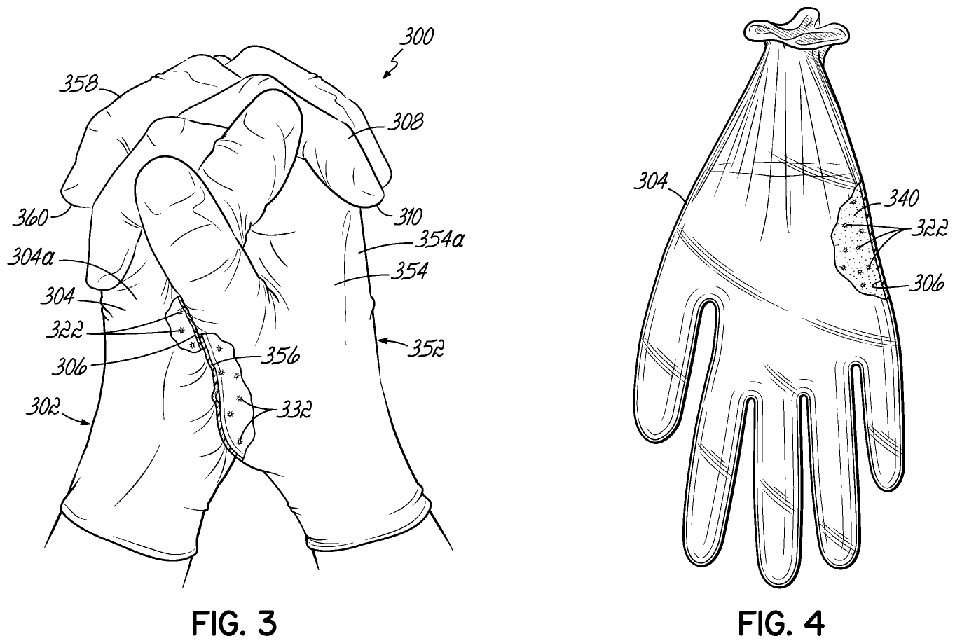

With reference now to FIGS. 1 and 3, a method 100 for testing an individual's hand cleansing effectiveness in accordance with an embodiment of the invention is shown. The method 100 includes, at step 102, donning a first glove 304 (FIG. 3) on a first hand 302 (FIG. 3) and, at step 104, optionally donning a second glove 354 (FIG. 3) on the other or second hand 352 (FIG. 3). To accomplish donning of the gloves, each hand 302, 352 can be inserted into its respective glove 304, 354 such that the hand is appropriately received therein. For example, as shown in FIG. 3, each tip 310 of each finger 308 of the first hand 302 and each tip 360 of each finger 358 of the second hand 352 can be received within corresponding finger holes of the first and second gloves 304, 354. Donning of the first and second gloves 304, 354 can be done in any order, with cleansing of the first hand 302 and/or second hand 352 to follow as discussed below. The gloves 304, 354 here may be standard gloves as known in the medical/healthcare industry. In one example, the gloves are sterile gloves, such as sterile surgical gloves, and may be latex, nitrile, rubber, or the like. The gloves 304, 354 initially may be packaged, individually or collectively, in appropriate packaging as known in the industry, for example, to maintain sterility of the gloves. When a user is ready, the glove(s) may need to be removed from their packaging. It is further contemplated here that the gloves may be fingerless such that hand hygiene testing of an individual is of the palms only. Also, the gloves may be of a mitt/mitten design such that there are no segregated or separate finger portions. And it is further yet contemplated that individual finger or fingertip sleeves (not shown) may be used for one or more fingers on one or both hands instead of or in conjunction with one or more gloves.

The method 100 next includes, at step 106, engaging the gloved first hand 302 and the gloved second hand 352. In one example, the palms of the hands 302, 352 can be pressed together, e.g., firmly. In another example, the fingers may be interlocked. It is contemplated here that only one hand 302, 352 may be gloved for testing purposes. As shown, the gloved hands 302, 352 are both pressed together with fingers interlocked. The pressing and/or interlocking may occur for a desired period of time, such as 10 seconds, so that a deposition of any microbes 322 can occur from the first and second hand 302, 352 onto an interior surface 306, 356 of the first and second glove 304, 354, respectively. In other examples, the pressing and/or interlocking may occur for more than or less than 10 seconds, such as for 5, 20, 30, 45, or 60 seconds, with even longer times contemplated, if so desired/needed.

The method 100 next includes, at step 108, removing the first glove 304 from the first hand 302 and the second glove 354 from the second hand 352. In one example, the gloves 304, 354 may be removed by pulling from the fingertips of the glove. The first glove 304 and the second glove 354 may be set aside in an environment such that the interior surfaces 306, 356 are not contaminated by outside substances, materials, or microbes. There generally is no concern about contaminating the outer surface of the gloves but care should be taken to avoid contaminating the interior surface 306, 356 of the gloves 304, 354. Thus, aseptic techniques should be used when removing (and donning) the gloves. In one example, the gloves 304, 354 should not be turned inside out.

After removing the gloves 304, 354, at step 109, the gloves 304, 354 may be placed together or separately in a container or package to properly contain them for sending to a lab for the desired testing. The container or package can generally include any known or standard type of container or package, including an envelope, bag (e.g., with closure tabs or twist tie closures), or baggies (e.g., zip lock), or the like. The container or package may be of a medical, sterilized, or sanitary type, as needed/necessary. The container or package can be marked accordingly, such as in a way to identify the glove(s) 304, 354 but that reduces or eliminates the risk that the tested individual can be identified by the lab. In one example, only one of the first or second gloves may be sent for testing. In another example, the gloves 304, 354 may be set aside, for later packaging, in an environment such that the interior surfaces 306, 356 are not contaminated by outside substances, materials, or microbes.

After the first glove 304 and the second glove 354 have been removed and optionally placed into their container or package, the method 100 can include, at step 111, cleansing the first hand 302 and/or the second hand 352. Cleansing can include hand washing and/or use of alcohol-based gels/liquids or other disinfectants, which can be followed by drying the hands 302, 352. In one example, cleansing can include washing the first hand 302 and the second hand 352, which may be done using the individual's ordinary technique. In some examples, cleansing the first hand 302 and the second hand 352 may include water and/or a disinfectant. In some examples, cleansing the first hand 302 and the second hand 352 includes the use of soap or soap and water. In some examples, the individual being tested uses a more thorough cleansing technique than his or her ordinary technique, for example, according to his or her employment protocol or policy. It is further contemplated that one hand may be cleansed differently than the other.

After cleansing step 111, the method 100 can include, at step 112 and with continuing reference to FIG. 3, donning a third glove 304a on the first hand 302 and, at step 114, optionally donning a fourth glove 354a on the second hand 352. It should be readily understood here that each of the first and second gloves 304, 352 are simply replaced with two new gloves 304a, 354a with one each being for the first and second hand 302, 352, respectively. Donning is as discussed above with each hand 302, 352 being inserted into its respective glove 304a, 354a such that the hand is appropriately received therein. Here again, the gloves 304a, 354a may be standard gloves as known in the medical/healthcare industry. In one example, the gloves are sterile gloves, such as sterile surgical gloves, and may be latex, nitrile, rubber, or the like. The type of glove used in these steps 111, 112 generally should be (but may not be) the same type of glove used in prior steps 102, 104 for testing consistency. The gloves 304a, 354a initially may be packaged, individually or collectively, in appropriate packaging as known in the industry, for example, to maintain sterility of the gloves. When a user is ready, the glove(s) may need to be removed from their packaging. It is further contemplated here that the gloves may be fingerless such that hand hygiene testing of an individual is of the palms only. Also, the gloves may be of a mitt/mitten design such that there are no segregated or separate finger portions. And it is further yet contemplated that individual finger or fingertip sleeves (not shown) may be used for one or more fingers on one or both hands instead of or in conjunction with one or more gloves.

The method 100 next can include, at step 115, engaging the gloved first hand 302 and the gloved second gloved 352. In one example, the palms of the hands 302, 352 are pressed together, e.g., firmly. In another example, the fingers may be interlocked. As shown, the gloved hands 302, 352 are both pressed together with fingers interlocked. The pressing and/or interlocking may occur for a desired period of time, such as 10 seconds, so that a deposition of any microbes 322 can occur from the first and second hands 302, 352 onto an interior surface of the third and fourth glove, respectively. In other examples, the pressing and/or interlocking may occur for more than or less than 10 seconds, such as for 5, 20, 30, 45 or 60 seconds, with even longer times contemplated, if so desired/needed.

The method 100 can further include, at step 116, removing the third and fourth glove 304a, 354a from the first and second hand 302, 352, respectively. In one example, the gloves 304a, 354a may be removed by pulling from the fingertips of the glove. The third glove 304a and the fourth glove 354a may be set aside in an environment such that the interior surfaces 306, 356 are not contaminated by outside substances, materials, or microbes. There generally is no concern about contaminating the outer surface of the gloves but care should be taken to avoid contaminating the interior surface 306, 356 of the gloves 304a, 354a. Thus, aseptic techniques should be used when removing (and donning) the gloves. In one example, the gloves 304a, 354a should not be turned inside out.

After removing the gloves 304a, 354a, at step 117, the gloves 304a, 354a may be placed together or separately in a container or package to properly contain them for sending to a lab for the desired testing. The container or package can generally include any known or standard type of container or package, including an envelope, bag (e.g., with closure tabs or twist tie closures), or baggies (e.g., zip lock), or the like. The container or package may be of a medical, sterilized, or sanitary type, as needed/necessary. The container or package can be marked accordingly, such as in a way to identify the glove(s) 304a, 354a but that reduces or eliminates the risk that the tested individual can be identified by the lab. In one example, only one of the third or fourth gloves 3041, 354a may be sent for testing. In another example, the gloves 304a, 354a may be set aside for later packaging in an environment such that the interior surfaces 306, 356 are not contaminated by outside substances, materials, or microbes.

Next, at step 118, all of the gloves 304, 354, 304a, 354a can be further packaged together or separately and sent to a testing facility, e.g., a laboratory, for testing. For example, the first, second, third, and fourth gloves 304, 354, 304a, and 354a can be collected and packaged together in a single container or package or in separate containers or packages to properly contain them for sending to a lab for the desired testing. The container or package can generally include any known or standard type of shipment container or package, including envelopes, bags, boxes, or the like. The container or package may be of a medical, sterilized, or sanitary type, as needed/necessary.

Thereafter, at step 120, the first glove 304, the second glove 354, the third glove 304a, and the fourth glove 354a can be subjected to testing at the desired lab, such as to determine the total viable aerobic bacteria. The gloves can be tested for a microbe presence by test methods known in the industry. Testing can include utilization of principles from ASTM 1174 and methodologies in the current edition of The United States Pharmacopeia NF, Rockville, Md., with proper aseptic techniques ideally being employed in all processing steps such as to avoid extrinsic microbial contamination. In one example, only one of the first or second gloves 304, 354 (e.g., the first glove 304) and one of the third or fourth gloves 304a, 354a (e.g., the corresponding third glove 304a) ultimately may be tested.

With specific reference now to FIG. 4, testing of the first set of gloves 304 and 354 (only one glove 304 shown) can include contacting the interior of glove 304 with an appropriate extraction medium 340. For example, the interior of glove 304 can be filled with polysorbate or a combination of polysorbate and lecithin (optionally including neutralizing agents), and the like, and further optionally agitated (e.g., shaking or vortexing) for a desired period of time and at a desired temperature to extract microbes 322 from the interior surface 306. In one example, 100 mL of extraction buffer, such as Dey Engley Neutralizing Broth, can be poured into glove 304. Then, the exterior of the glove can be massaged for about 1 minute so that the interior of the glove is rubbed against itself, including manipulation of the fingers, thumb, and/or palm. In addition, the top of the glove may be sealed and shaken with moderate intensity for about 1 minute. The microbes 322 can exhibit an affinity to the extraction medium 340. Thereafter, in one example, an aliquot can be extracted from the first glove 304 and can be added to the second glove 354, then the second glove 354 can be subjected to the above process. For example, the interior of the second glove 354 can be filled with an appropriate extraction medium 340, e.g., polysorbate or a combination of polysorbate and lecithin (optionally including neutralizing agents), and the like, and optionally agitated for a desired period of time and at a desired temperature to extract microbes 322 from the interior surface 306. In another example, no aliquot from the first glove 304 is placed into the second glove 354 and, instead, the gloves 304, 354 (including gloves 304a, 354a) may be individually processed and tested. The same techniques above then can be used/repeated for gloves 304a and 354a.

In addition and aside from the above process of filling the interior of the gloves 304, 354, 304a, 354a with an appropriate extraction medium, it is contemplated that the gloves 304, 354, 304a, 354a could be flipped inside out or could be cut up and all or some of the pieces thereof taken and subjected to traditional extraction techniques, including contacting the pieces with a desirable volume of extraction medium inside a container, such as a beaker, flask, or test tube, for example, and optionally agitating for a desired period of time and at a desired temperature to extract microbes 322 from the interior surface 306.

After the desired extraction method is completed, aliquots of the extracted media can be taken from the desired/specified glove 304, 354, 304a, 354a to enumerate microbiological contaminates in ways known to those in the art to identify, for example, aerobic microorganisms. In one example, a minimum of 23.0 mL of an aliquot from the second glove 354 and the fourth glove 354a can be taken and added to their own corresponding and appropriate-sized container, such as a 50 mL conical vial, and standard pour plate techniques utilized. In one example, 1:10 serial dilutions of each of the samples are prepared. For the initial dilution, 10.0 mL from each of the conical vial aliquots can be placed across three Petri dishes, which is conducted in duplicate. This represents the 10⁻¹ dilution. For subsequent dilutions, 1.0 mL can be plated into sterile Petri dishes, which is conducted in duplicate. About 12 to 15 mL of tempered (45-47° C.) Microbial Content Test Agar can be poured into the Petri dishes. The samples and the media are mixed carefully, the plates allowed to solidify, and incubated (e.g., at 32.5±2.5° C. for 3 to 5 days) to evaluate total viable aerobic bacteria.

While different test methods may be used to test one or more of the gloves, in one example, the same test methods are utilized for each glove for the sake of direct comparison. The testing of each glove can include, for example, comparing the microbes 322 extracted from the first and/or second glove 304, 354 with the microbes extracted from the third and/or fourth glove 304a, 354a to assess the effectiveness of the individual's hand cleansing techniques. In some examples, the testing may include evaluating how many of a particular microbe are present in the microbe sample extracted from each glove or how many total microbes are present in the microbe samples extracted from each of the gloves. Generally speaking, the test data can be compared and the results evaluated to assess the effectiveness of each individual's hand cleansing technique(s). In one example, a Quebec Colony Counter counts each bacterial colony and records the count for all countable dilutions. Dilutions yielding counts <25 or >250 cfu/mL may be reported as the exact number of colonies counted, with estimations maybe required if no dilution produces counts between 25 and 250 CFU. Sample controls are run as appropriate for the above testing.

The method 100 may be repeated within a predetermined time period to evaluate the effects of an individual cleansing his or her hands more frequently or less frequently throughout the predetermined amount of time. In some examples, the predetermined amount of time may be an hour, an eight hour work shift, a day, or a week. In one example, the testing may be done at random. In other examples, the method 100 includes testing a plurality of individuals simultaneously or a single individual. In other examples, the method 100 includes dividing the plurality of individuals being tested into at least a first group and a second group for comparative analysis. In other examples, the first group may be given a first kind of disinfectant or soap to use when cleansing the first and second hand 302, 352, and the second group may be given a second kind of disinfectant or soap, or neither, to use when cleansing the first and second hand 302, 352. A comparison of the microbes extracted from the gloves of the first group and the microbes extracted from the gloves of the second group may assist in determining whether a specific disinfectant or soap and/or cleansing technique is superior at eliminating microbes, for example.

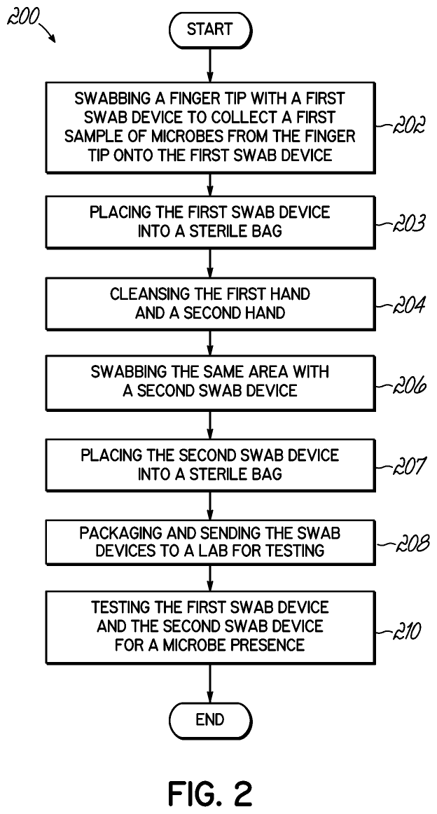

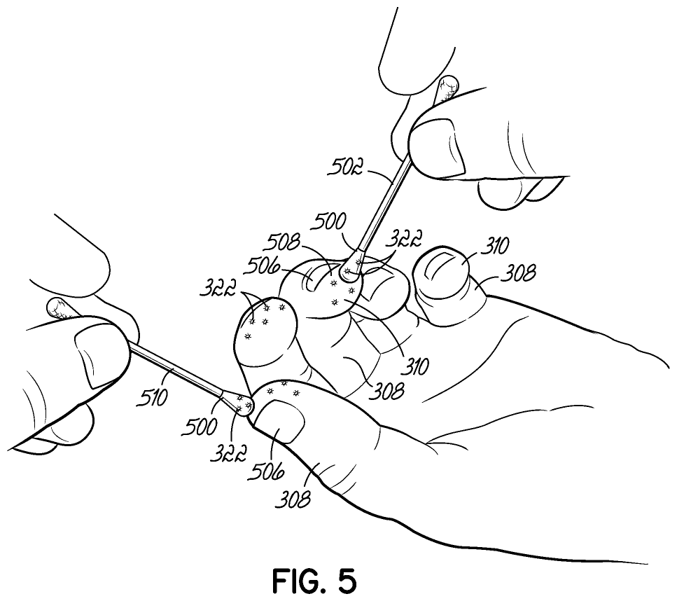

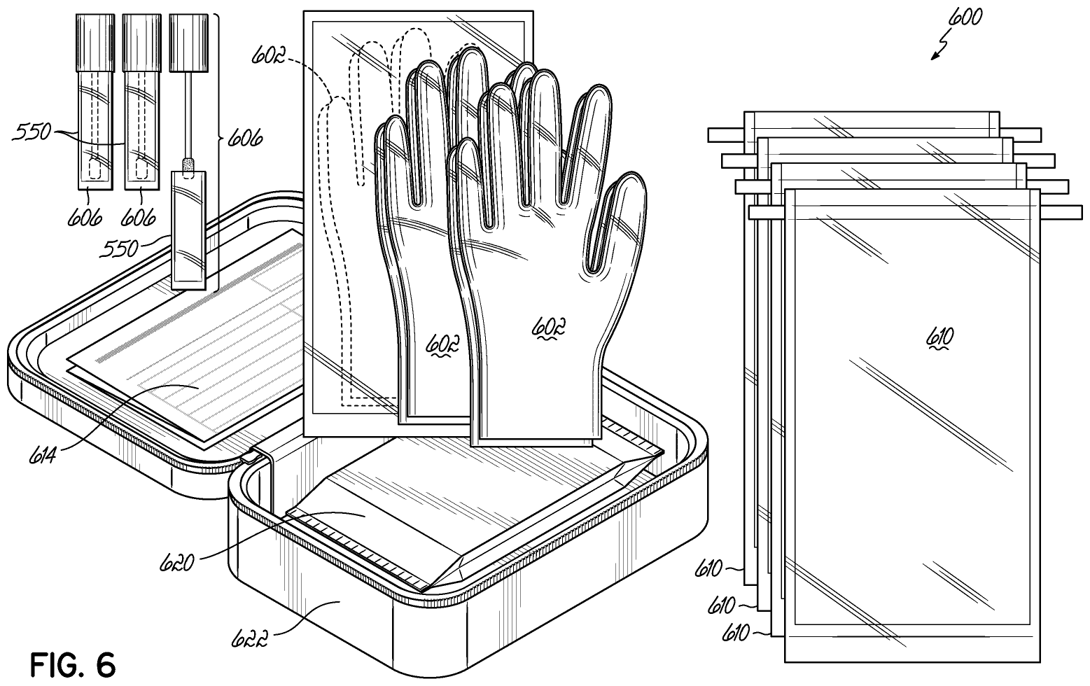

With reference now to FIGS. 2 and 5, a method 200 for testing an individual's hand cleansing effectiveness in accordance with another embodiment of the invention is shown. This method 200 may be performed altogether separately from method 100 or combined therewith, either in whole or in part and vice versa. The method 200 includes, at step 202, swabbing at least one fingertip 310 (FIG. 5) on a first hand with a tip 500 of a first swab device 502 (FIG. 5) to collect a first sample of microbes 322 (FIG. 5) from the fingertip 310 onto the first swab device 502. In one example, the first swab device 502 may swab a fingertip area 508 that is located at and/or under the fingernail 506 of the same fingertip. In another example, the first sample of microbes includes microbes collected from each fingertip 310 and/or fingertip area 508 that is located at and/or under the fingernail 506 of the same fingertip on the first hand 302 (FIG. 3). In such examples, a single swab device or multiple swab devices may be used in the collection of the first sample of microbes 322. For example, a plurality of first swabbing devices may be used such that a different swab device is used to collect a sample of microbes from each fingertip 310 and/or fingertip area 508 that is located at and/or under the fingernail 506 of the same fingertip. Also, the swabbing of the fingertip 310 may occur on the first hand 302 only, or on both the first hand 302 and the second hand 352 (FIG. 3). In examples where swabbing occurs on the second hand 352 in addition to the first hand 302, the method 200 similarly may include the first swab device 502 swabbing at least one fingertip 360 (FIG. 3) and/or fingertip area of the second hand 352. Alternatively, the method 200 may include a different swab device swabbing at least one fingertip 360 and/or fingertip area 508 of the second hand 352, as discussed above with respect to the first hand 302. Similarly, in such examples, a single swab device or multiple swab devices may be used in the collection of the sample of microbes 322 collected from the second hand 352. The swab device 502 can be a standard dry swab, which may be sterile and may be associated with a corresponding test tube container 550 (FIG. 6) such that the swab and test tube container together can define the swab device 502. The test tube container/dry swab combination are known in the medical/healthcare industry and may be of a medical, sterilized, or sanitary type. In one example, the swab device 502 is not an ATP type swab.

After swabbing at least one fingertip 310 on a first hand with the first swab device 502, at step 203, the swab device 502 (with or without an associated test tube 550) may be placed in a container or package or, if there are multiple swab devices, placed therein together or in separate containers or packages to properly contain them for sending to a lab for the desired testing. The container or package can generally include any known or standard type of container or package, including its own test tube, an envelope, bag (e.g., with closure tabs or twist tie closures), or baggies (e.g., zip lock), or the like. The container or package may be of a medical, sterilized, or sanitary type, as needed/necessary. The container or package can be marked accordingly, such as in a way to identify the swab device(s) 502 but that reduces or eliminates the risk that the tested individual can be identified by the lab. In another example, the swab device 502 may be set aside for later packaging in an environment such that the swab device 502 is not contaminated by outside substances, materials, or microbes.

After the swab device 502 has been placed into its container or package, the method 200 can further include, at step 204, cleansing at least the swabbed hand(s) 302, 352 of the individual being tested. Cleansing can include hand washing and/or use of alcohol-based gels/liquids or other disinfectants, which can be followed by drying the hands 302, 352. In one example, cleansing can include washing the first hand 302 and the second hand 352, which may be done using the individual's ordinary technique. In some examples, cleansing the first hand 302 and the second hand 352 may include water and/or a disinfectant. In some examples, cleansing the first hand 302 and the second hand 352 includes the use of soap or soap and water. In some examples, the individual being tested uses a more thorough cleansing technique than his or her ordinary technique, for example, according to his or her employment protocol or policy. It is further contemplated that one hand may be cleansed differently than the other.

After the cleansing step 204, the method 200 can include, at step 206, swabbing the same fingertip(s) 310 and/or fingertip area(s) 508 previously swabbed prior to cleansing, with a second or additional swab device 510 to collect a second or additional sample of microbes 322 onto the additional swab device 510. In another example, the method 200 can include, at step 206, swabbing less than or more than the same fingertip(s) 310 and/or fingertip area(s) 508 previously swabbed prior to cleansing. A single swab device or multiple swab devices may be used in the collection of the additional sample(s) of microbes 322. For example, a plurality of second swabbing devices may be used such that one or more distinct second swab devices are used to collect a sample of microbes from each fingertip 310 and/or fingertip area 508 on one or both hands. Here again, swab device 510 can be a standard dry swab, which may be sterile and may be associated with a corresponding test tube container 550 (FIG. 6) such that the swab and test tube container together can define the swab device 502. The test tube container/dry swab combination is known in the medical/healthcare industry and may be of a medical, sterilized, or sanitary type. In one example, the swab device 510 is not an ATP type swab.

After swabbing with the second swab device 510, at step 207, the swab device 502 (with or without an associated test tube 550) may be placed in a container or package or, if there are multiple swab devices, placed therein together or in separate containers or packages to properly contain them for sending to a lab for the desired testing. The container or package can generally include any known or standard type of container or package, including its own test tube, an envelope, bag (e.g., with closure tabs or twist tie closures), or baggies (e.g., zip lock), or the like. The container or package may be of a medical, sterilized, or sanitary type, as needed/necessary. The container or package can be marked accordingly, such as in a way to identify the swab device(s) 510 but that reduces or eliminates the risk that the tested individual can be identified by the lab. In another example, the swab device 510 may be set aside for later packaging in an environment such that the swab device 510 is not contaminated by outside substances, materials, or microbes.

Next, at step 208, the swab devices 502, 510 can be further packaged together or separately and sent to a testing facility, e.g., a laboratory, for testing. For example, the first and second swab devices 502, 510 can be collected and packaged together in a single container or package or in separate containers or packages to properly contain them for sending to a lab for the desired testing. The container or package can generally include any known or standard type of shipment container or package, including envelopes, bags, boxes, or the like. The container or package may be of a medical, sterilized, or sanitary type, as needed/necessary.

Thereafter, at step 210, the first and second swab devices 502, 510 can be subjected to testing at the desired lab, such as to determine the total viable aerobic bacteria. The swab devices 502, 510 can be tested for a microbe presence by test methods known in the industry. Testing can include utilization of principles from ASTM 1174 and methodologies in the current edition of The United States Pharmacopeia NF, Rockville, Md., with proper aseptic techniques ideally being employed in all processing steps such as to avoid extrinsic microbial contamination.

In one example, testing of the swab devices 502, 510 can include separately subjecting, such as contacting, at least the swab tip 500 of the swab devices with an appropriate extraction medium 340 (FIG. 4), e.g., polysorbate or a combination of polysorbate and lecithin (optionally including neutralizing agents), and the like. Immersion in the extraction medium within a container, such as a beaker, flask, or test tube, for example, and optional agitation (e.g., shaking or vortexing) of the swab tip of the swab device for a desired period of time and at a desired temperature to extract microbes 322 therefrom may be required. In one example, 9 mL of extraction buffer, such as Dey Engley Neutralizing Broth, can be poured into a vial that includes swab device 502 (or at least the tip 500 thereof). The vial can be vortexed for about 60 seconds at ambient temperature. The same techniques can be employed for swab device 510. In addition and aside from the above process of immersing at least the swab tip 500 in an appropriate extraction medium, it is contemplated that the swab device 502, 510 itself or tip 500 could be cut up or pulverized with all or some of the cut or pulverized material thereof taken and subjected to traditional extraction techniques, including contacting the material with a desirable volume of extraction medium inside a container, such as a beaker, flask, or test tube, for example, and optionally agitating (e.g., shaking or vortexing) for a desired period of time and at a desired temperature to extract microbes 322 therefrom.

After the desired extraction method is completed, aliquots of the extracted media can be taken from the desired/specified swab device 502, 510 to enumerate microbiological contaminates in ways known to those in the art by using standard pour plate techniques to identify, for example, aerobic microorganisms. In one example, 1:10 serial dilutions of the sample are prepared. For the initial dilution, 1.0 mL from the vial aliquot can be placed across three Petri dishes, which is conducted in duplicate. This represents the 10⁻¹ dilution. For subsequent dilutions, 1.0 mL can be plated into sterile Petri dishes, which is conducted in duplicate. About 12 to 15 mL of tempered (45-47° C.) Microbial Content Test Agar can be poured into the Petri dishes. The samples and the media are mixed carefully, the plates allowed to solidify, and incubated (e.g., at 32.5±2.5° C. for 3 to 5 days) to evaluate total viable aerobic bacteria. In other test methods, the swabbed tips 500 of the swab devices 502, 510 may be subjected to corresponding agar plates in accordance with standard industry test methods known in the art to culture, enumerate, and identify microbiological contaminates.

While different test methods may be used to test one or more of the swab devices, in one example, the same test methods are utilized for each swab device for the sake of direct comparison. The testing of each swab device can include, for example, comparing the microbes 322 extracted from the first swab device 502 with the microbes extracted from the second swab device 510 to assess the effectiveness of the individual's hand cleansing techniques. In some examples, the testing may include evaluating how many of a particular microbe are present in the microbe sample extracted from each swab device or how many total microbes are present in the microbe samples extracted from each of the swab devices. Generally speaking, the test data can be compared and the results evaluated to assess the effectiveness of each individual's hand cleansing technique(s). In one example, a Quebec Colony Counter counts each bacterial colony and records the count for all countable dilutions. Dilutions yielding counts <25 or >250 cfu/mL may be reported as the exact number of colonies counted, with estimations maybe required if no dilution produces counts between 25 and 250 CFU. Sample controls are run as appropriate for the above testing.

The method 200 may be repeated within a predetermined time period to evaluate the effects of an individual cleansing his or her hands more frequently or less frequently throughout the predetermined amount of time. In some examples, the predetermined amount of time may be an hour, an eight hour work shift, a day, or a week. In one example, the testing may be done at random. In other examples, the method 200 includes testing a plurality of individuals simultaneously or a single individual. In other examples, the method 200 includes dividing the plurality of individuals being tested into at least a first group and a second group for comparative analysis. In other examples, the first group may be given a first kind of disinfectant or soap to use when cleansing the first and second hand 302, 352, and the second group may be given a second kind of disinfectant or soap, or neither, to use when cleansing the first and second hand 302, 352. A comparison of the microbes extracted from the swab device(s) of the first group and the microbes extracted from the swab device(s) of the second group may assist in determining whether a specific disinfectant or soap and/or cleansing technique is superior at eliminating microbes, for example.

In another embodiment, all or a portion of the test method 100 of FIG. 1, in which gloves 304, 354, 304a, 354a may be utilized to test hand hygiene techniques, can be combined with all or a portion of the test method 200 of FIG. 2, in which the first and second swab devices 502, 510 are utilized to test hand hygiene techniques. In one example, swabbing of the fingertip(s) and/or fingertip area(s) with swab device 502 prior to cleansing of the hands 302, 352 can be omitted altogether such that only swab device 510 is used to collect a sample(s) of microbes 322 thereon after cleansing of the hands, and such swab testing can be combined with the entirety of the test method of FIG. 1. For example, the swabbing of the fingertip(s) and/or fingertip area(s) with swab device 510 can occur before or after the second set of gloves 304a, 354a are used on the cleansed hands 302, 352 for testing of hand hygiene techniques. In this example, the packaging and sending of gloves 304, 354, 304a, 354a and swab device(s)510 may be done together or separately such that the testing thereof may be performed at a lab, as discussed above. The methods and testing for such contemplated combined test method are as discussed above for the separate test methods 100, 200. In another example, the donning of the first and second gloves 304, 354 prior to cleansing of the hands 302, 352 can be omitted altogether such that only gloves 304a, 354a are used to collect a sample(s) of microbes 322 thereon after cleansing of the hands, and such glove testing can be combined with the entirety of the test method of FIG. 1. For example, the use of gloves 304a, 354a can occur before or after the second swab device 510 is used on the cleansed hands 302, 352 for testing of hand hygiene techniques. Here again, in this example, the packaging and sending of gloves 304a, 354a and swab devices 502, 510 may be done together or separately such that the testing thereof may be performed at a lab, as discussed above. The methods and testing for such contemplated combined test method are as discussed above for the separate test methods 100, 200. Other variations and combinations can be contemplated as well.

With reference now to FIG. 6, a hand hygiene test kit 600 can be provided that can include all of the items necessary to aid in carrying out one or both of the methods 100, 200 separately or combined in ways discussed hereinabove, for example. As shown, the test kit 600 can include three sets/pairs of or six total gloves 602, with four of the gloves 602 corresponding, for example, to first glove 304, second glove 354, third glove 304a, and fourth glove 354a. The remaining pair of or two gloves can be designated for demonstration or practice purposes, or as a backup pair in the event an additional pair is required. In one example, the test kit 600 simply can include two sets or pairs of gloves 602, with one glove from each pair capable of being donned on an individual's left hand and the other glove capable of being donned on the individual's right hand. In another example, only one pair or more than three pairs of gloves 602 can be provided. In another example, an odd number of gloves 602, including one glove, may be provided. As discussed above, the gloves 602 may be standard gloves as known in the medical/healthcare industry. In one example, the gloves are sterile gloves, such as sterile surgical gloves, and may be latex, nitrile, rubber, or the like. The gloves 602 initially may be packaged, individually or collectively, in appropriate packaging as known in the industry, for example, to maintain sterility of the gloves. To that end, when a user is ready, the glove(s) may need to be removed from their packaging.

The test kit 600 alternatively or additionally can include one or more swab devices 606. As shown, the test kit 600 can include three swab devices 606, with two of three corresponding, for example, to first swab device 502 and second swab device 510. The remaining swab device 606 can be designated for demonstration or practice purposes, or as a backup in the event an additional one is required. In one example, the kit 600 simply can include two swab devices 606, where one swab device can be used on one hand and the other swab device can be used on the other hand. In another example, only one or more than three swab devices 606 can be provided. As discussed above, the swab device 606 can be a standard dry swab, which may be sterile and may be associated with a corresponding test tube container 550 such that the swab and test tube container together can define swab device 606. The test tube container/dry swab combination are known in the medical/ healthcare industry and may be of a medical, sterilized, or sanitary type. In one example, the swab device 606 is not an ATP type swab.

With continuing reference to FIG. 6, the test kit 600 can further optionally include one or more containers or packages 610, such as an envelope, bag (e.g., with closure tabs or twist tie closures), or baggies (e.g., zip lock), or the like, for securely receiving the gloves 602 and/or swab devices 606 (with or without a corresponding test tube 550) after use. In one example, as shown, the test kit 600 includes at least four bags 610 (optionally with twist tie closures) with each of two bags capable of receiving one pair of gloves 602, such as first and second gloves 304, 354 and third and fourth gloves 304a, 354a, respectively, and the remaining two bags each capable of receiving a swab device 606, such as two different second swab devices 510 where each of the second swab devices 510 is used on a single hand after the hands have been cleansed, as discussed above when test method 200 has been combined, in part, with test method 100. In one example, the test kit 600 simply can include two containers or packages 610, where one container or package 610 receives one set of gloves 602 or one swab device 606 and the other container or package 610 receives another set of gloves 602 or another swab device 606. In other examples, the kit can include one, three, or more than 4 containers or packages 610.

The test kit 600 also can include other items including, for example, a sample submission form(s) 614 and one or more ice packs 620 (e.g., self-activated or standard ice packs) to accompany the submitted samples (e.g., the gloves 602 and/or swab(s) 606). The ice pack(s) 620 can help preserve the sample(s) when sent, such as via carrier, to the lab for testing. The test kit 600 also can include its own container 622 for storing and shipping the test kit items 602, 606, 610, 614, 620 to a user or facility and for storing and shipping the test samples 304, 354, 304a, 354a, 502, 510 to the lab. The test kit 600 may be shipped with other test kits and cases to a user or facility and may be colored or color coded, e.g., red, to set it apart from other test kits/cases that may be shipped along with it.

While the present invention has been illustrated by a description of various embodiments and while these embodiments have been described in considerable detail, it is not the intention of the applicant to restrict or in any way limit the scope of the appended claims to such detail. By way of example, the methods and test kits discussed herein additionally or alternatively may be used to assess the rate of occupational soiling of an individual's/employee's hand(s) and can include, for example, testing at the start of a shift and at some point or intervals thereafter, then comparing and contrasting the test results. The methods and test kits also may be used, for example, to assess occupational hand contamination risks such as by time/job function, with the test results potentially utilized to adjust the frequency of hand washing, the need for an employee(s) to use protective gloves, etc. Also, in addition to healthcare facilities, textile service providers, and laundries, it should be realized that the methods and test kits herein can be utilized across many industries and for varying applications including, for example, manufacturing facilities (where cleanliness is desirable), such as clean rooms, food processing, and medical device manufacturing, service providers such as restaurants and extended care facilities, public education where controlling hand hygiene is desirable and for teaching kids about hand hygiene, and academia such as to study hand hygiene practices, etc. Additional advantages and modifications will readily appear to those skilled in the art. Thus, the invention in its broader aspects is therefore not limited to the specific details, representative apparatus and method and illustrative example shown and described. Accordingly, departures may be made from such details without departing from the spirit or scope of applicant's general inventive concept.

What is claimed is:

1. A method for assessing the effectiveness of an individual's hand cleansing technique, the method comprising:
donning a first glove on an individual's hand;
engaging the gloved hand and a second hand of the individual for a desired period of time;
removing the first glove from the hand;
after removing the first glove, cleansing the hand;
thereafter, donning a second glove on the cleansed hand;
engaging the gloved hand having the second glove and the second hand of the individual for a desired period of time; and
removing the second glove from the hand,
wherein an interior of the first glove and the second glove is ready to be tested for a microbe presence so that test data can be evaluated to assess the effectiveness of the individual's hand cleansing technique.

2. The method of claim 1 further comprising:
after cleansing the hand, swabbing at least one fingertip on the individual's cleansed hand with a first swab device to collect a first sample from the fingertip, wherein the first sample is ready to be tested for a microbe presence so that test data can be evaluated to assess the effectiveness of the individual's hand cleansing technique.

3. A method for assessing the effectiveness of an individual's hand cleansing technique, the method comprising:
donning a first glove on an individual's first hand and a second glove on a second hand of the individual;
engaging the gloved first hand and the gloved second hand for a desired period of time;
removing the first and second gloves from the first and second hands;
after removing the first and second gloves, cleansing the first and second hands;
thereafter, donning a third glove on the first cleansed hand and donning a fourth glove on the second cleansed hand;
engaging the gloved first hand having the third glove and the gloved second hand having the fourth glove for a desired period of time; and
removing the third and fourth gloves from the first and second hands,
wherein an interior of at least one of the first or second gloves and one of the third or fourth gloves is ready to be tested for a microbe presence so that test data can be evaluated to assess the effectiveness of the individual's hand cleansing technique.

4. The method of claim 3 wherein engaging the gloved first hand and the gloved second hand and/or engaging the gloved first hand having the third glove and the gloved second hand having the fourth glove includes pressing the palms together and/or interlocking the fingers.

5. The method of claim 3 wherein the gloved first hand and the gloved second hand and/or the gloved first hand having the third glove and the gloved second hand having the fourth glove are engaged for at least ten seconds.

6. The method of claim 3 wherein cleansing the first and second hands includes cleansing the first and second hands followed by drying the first and second hands.

7. The method of claim 3 wherein cleansing the first and second hands includes washing the first and second hands and/or using alcohol based gels or liquids or other disinfectants to cleanse the first and second hands.

8. The method of claim 3 further comprising, after removing the third and fourth gloves from the first and second hands, packaging and sending at least one of the first or second gloves and one of the third or fourth gloves to a laboratory, wherein an interior of at least one of the first or second gloves and one of the third or fourth gloves is ready to be tested for a microbe presence so that test data can be evaluated to assess the effectiveness of the individual's hand cleansing technique.

9. The method of claim 3 wherein the individual is an employee of a textile service provider or a laundry facility.

10. The of claim 3 further comprising testing at least one of the first or second gloves and one of the third or fourth gloves for a microbe presence and comparing test data to assess the effectiveness of the individual's hand cleansing technique.

11. The method of claim 3 further comprising:
after cleansing the first and second hands, swabbing at least one fingertip on the individual's cleansed first hand with a first swab device to collect a first sample from the fingertip and swabbing at least one fingertip on the individual's cleansed second hand with a second swab device to collect a second sample from the fingertip, wherein the first and second samples are ready to be tested for a microbe presence so that test data can be evaluated to assess the effectiveness of the individual's hand cleansing technique.

12. A method for assessing the effectiveness of an individual's hand cleansing technique, the method comprising:
swabbing at least one fingertip on an individual's first hand with a first swab device to collect a first sample from the fingertip;
cleansing the first hand of the individual; and
thereafter, swabbing at least the same fingertip on the individual's cleansed first hand with a second swab device to collect a second sample from the fingertip,
wherein the first and second sample of the first and second swab devices are ready to be tested for a microbe presence so that test data can be evaluated to assess the effectiveness of the individual's hand cleansing technique.

13. The method of claim 12 wherein swabbing at least one fingertip on an individual's first hand and/or swabbing the same fingertip on the individual's cleansed first hand with a second swab device includes swabbing at and/or under the fingernail of the fingertip with the swab device to collect the sample from the fingertip.

14. The method of claim 12 wherein cleansing the first hand includes cleansing the first hand followed by drying the first hand.

15. The method of claim 12 wherein the individual is an employee of a textile service provider or a laundry facility.

16. The method of claim 12 wherein swabbing at least one fingertip on an individual's first hand with a first swab device includes swabbing each fingertip on an individual's first hand with the first swab device to collect a first sample and wherein swabbing the same fingertip includes swabbing the same fingertips on the individual's cleansed first hand with a second swab device to collect a second sample from the fingertips.

17. The method of claim 12 wherein swabbing at least one fingertip on an individual's first hand with a first swab device includes swabbing at least on fingertip on the first hand and at least one fingertip on a second hand of the individual to collect a first sample and wherein swabbing the same fingertip includes swabbing the same fingertips on the individual's first and second hand with a second swab device to collect a second sample from the fingertips.

18. The method of claim 12 further comprising, after swabbing the same fingertip on the individual's cleansed first hand with the second swab device, packaging and sending the first and second swab devices to a laboratory, wherein the first and second swab devices are ready to be tested for a microbe presence so that test data can be evaluated to assess the effectiveness of the individual's hand cleansing technique.

19. The method of claim 12 further comprising testing the first sample of the first swab device and the second sample of the second swab device for a microbe presence and comparing test data to assess the effectiveness of the individual's hand cleansing technique.

20. A test kit for assessing the effectiveness of an individual's hand cleansing technique, the kit comprising:
at least two pairs of gloves, with one glove from each pair capable of being donned on an individual's left hand and the other glove capable of being donned on the individual's right hand, and/or
at least a first and second swab device, with each swab device configured to swab at least one fingertip on an individual's first hand and collect a sample from the fingertip; and
one or more containers or packages for receiving the gloves and/or swab devices after use by the individual, wherein the gloves and/or swab devices can be sent to a testing facility for testing to assess the effectiveness of an individual's hand cleansing technique.

21. The kit of claim 21 further comprising one or more sample submission forms.

22. The kit of claim 21 further comprising one or more ice packs.

23. The kit of claim 21 further comprising a container for storing and shipping the at least two pairs of gloves and/or first and second swab device, and the one or more containers or packages for receiving the gloves and/or swab devices.

24. The kit of claim 21 wherein the kit comprises the at least two pairs of gloves and the at least first and second swab device, and the one or more containers or packages for receiving the gloves and swab devices after use by the individual, wherein the gloves and swab devices can be sent to a testing facility for testing to assess the effectiveness of an individual's hand cleansing technique.

25. The kit of claim 21 wherein the kit comprises three pairs of gloves, a first, second, and third swab device, and four containers or packages.
